(19) United States
(12) Patent Application Publication
(10) Pub. No.: US 2020/0069696 A1
(43) Pub. Date: Mar. 5, 2020

(54) OPHTHALMIC INJECTABLE FORMULATION PREPARING AND OCULOPATHY TREATING AND PREVENTING

(71) Applicant: Yunxiang LIU, San Diego, CA (US)

(72) Inventor: Yunxiang LIU, San Diego, CA (US)

(21) Appl. No.: 16/553,739

(22) Filed: Aug. 28, 2019

Related U.S. Application Data
(60) Provisional application No. 62/725,064, filed on Aug. 30, 2018.

(51) Int. Cl. A61K 31/55; A61K 9/16; A61K 9/127; A61K 9/06; A61K 9/00; A61K 31/222; A61K 31/44

(52) U.S. Cl. CPC A61K 31/55; A61K 9/16; A61K 9/127; A61K 31/44; A61K 9/0024; A61K 9/0048; A61K 31/222; A61K 9/06

(57) ABSTRACT

Disclosed are an ophthalmic injectable sustained-release formulation, a process for preparing the same and a method for treating and preventing oculopathy with the same. The ophthalmic injectable sustained-release formulation comprises a delivery system and a pharmaceutically acceptable excipient, wherein the delivery system is selected from the group consisting of microspheres, microcapsules, microparticles, liposomes, multivesicular liposomes, nanocrystals and nanoparticles.

### CROSS-REFERENCE TO RELATED APPLICATIONS

The present application claims priority to and the benefit of U.S. Provisional Application No. 62/725,064, filed Aug. 30, 2018, the disclosure of which is hereby incorporated by reference in its entirety.

### FIELD

The present disclosure generally relates to the medical field. In particular, the present disclosure relates to the ophthalmic field.

### BACKGROUND

Eyelids consist of thin folds of skin, muscle, and connective tissue. The eyelids protect the eyes and spread tears over the front of the eyes. The inside of the eyelids is lined with the conjunctiva of the eyelid (the palpebral conjunctiva), and the outside of the lids are covered with the body's thinnest skin. Some common eyelid disorders include the following: stye, blepharitis, chalazion, entropion, ectropion, eyelid edema, eyelid tumors and myasthenia gravis.

The main treatment for eyelid disorders is currently by administration oral preparation or eyedrops. However, unwanted systemic side effects can often occur with administration oral preparation, including nausea/vomiting, diarrhea, stomach pain, increased salivation and tearing, irregular heartbeat, restlessness, anxiety, muscle twitching or tremor, blurred vision, and difficulty breathing. In addition, dosing with oral preparation or eyedrops is multiple times a day, which can negatively impact quality of life and reduce compliance.

### BRIEF DESCRIPTION

In one aspect, the disclosure relates to an ophthalmic injectable sustained-release formulation, comprising a delivery system and a pharmaceutically acceptable excipient, wherein the delivery system is selected from the group consisting of microspheres, microcapsules, microparticles, liposomes, multivesicular liposomes, nanocrystals and nanoparticles.

In another aspect, the disclosure relates to a process for preparing an ophthalmic injectable sustained-release formulation comprising a delivery system and a pharmaceutically acceptable excipient,

wherein the delivery system is selected from the group consisting of microspheres, microcapsules, microparticles, liposomes, multivesicular liposomes, nanocrystals and nanoparticles; and

wherein the process comprises

preparing the microspheres via emulsion solvent evaporation, double emulsion solvent evaporation, emulsification-chemical cross-linking method, emulsion-direct heat cross-linking method, spray drying, phase separation, SCF (supercritical fluid), ultrasonic atomization, electro spraying, hot melt extrusion, or polymer-alloys method.

In yet another aspect, the disclosure relates to a process for preparing an ophthalmic injectable sustained-release formulation comprising a delivery system and a pharmaceutically acceptable excipient,

wherein the delivery system is selected from the group consisting of microspheres, microcapsules, microparticles, liposomes, multivesicular liposomes, nanocrystals and nanoparticles; and

wherein the process comprises

preparing the liposomes via thin-film hydration, sonication, membrane extrusion, REV (reverse phase evaporation), ether injection, ethanol injection, freezing thawing, French press method, multiple emulsion, SRPE (supercritical fluid reverse phase evaporation), freeze drying of double emulsions, or ion gradient.

In still another aspect, the disclosure relates to a process for preparing an ophthalmic injectable sustained-release formulation comprising a delivery system and a pharmaceutically acceptable excipient,

wherein the delivery system is selected from the group consisting of microspheres, microcapsules, microparticles, liposomes, multivesicular liposomes, nanocrystals and nanoparticles; and

wherein the process comprises

preparing the multivesicular liposomes via double emulsion solvent evaporation.

In yet another aspect, the disclosure relates to a method for treating and preventing oculopathy, comprising administering an ophthalmic injectable sustained-release formulation to a subpalpebral conjunctiva plane just superior to a superior tarsal border across a horizontal width of an upper eyelid of an affected eye of a subject in need thereof,

wherein ophthalmic injectable sustained-release formulation comprises a delivery system and a pharmaceutically acceptable excipient, and

wherein the delivery system is selected from the group consisting of microspheres, microcapsules, microparticles, liposomes, multivesicular liposomes, nanocrystals and nanoparticles.

### DETAILED DESCRIPTION

In the following description, certain specific details are included to provide a thorough understanding for various disclosed embodiments. One skilled in the relevant art, however, will recognize that the embodiments may be practiced without one or more these specific details, or with other methods, components, materials, etc.

Unless the context required otherwise, throughout the specification and claims which follows, the term “comprise” and variations thereof, such as “comprises” and “comprising” are to be construed in an open, inclusive sense, which is as “include, but not limited to”.

Reference throughout this specification to “one embodiment”, or “an embodiment”, or “in another embodiment”, or “in some embodiments” means that a particular referent feature, structure or characteristic described in connection with the embodiments is included in at least one embodiment. Therefore, the appearance of the phrases “in one embodiment”, or “in the embodiment”, or “in another embodiment”, or “in some embodiments” in various places throughout this specification are not necessarily all referring to the same embodiment. Moreover, the particular features, structures or characteristics may be combined in any suitable manner in one or more embodiments.

It should be noted that, as used in this specification and the appended claims, the singular forms “a”, “an” and “the” include plural referents unless the context clearly stated otherwise. Therefore, for example, a reaction comprising “a pharmaceutically acceptable excipient” comprises one pharmaceutically acceptable excipient, two or more pharmaceutically acceptable excipients.

In one aspect, the disclosure relates to an ophthalmic injectable sustained-release formulation, comprising a delivery system and a pharmaceutically acceptable excipient, wherein the delivery system is selected from the group consisting of microspheres, microcapsules, microparticles, liposomes, multivesicular liposomes, nanocrystals and nanoparticles.

In some embodiments of the present disclosure, the ophthalmic injectable sustained-release formulation can be in the form of solution, suspension, or gel.

The exemplary pharmaceutically acceptable excipients that can be used in the present disclosure include, but not limited to, suspending agent, surfactant, matrix, stabilizer and isosmotic adjusting agent.

The exemplary suspending agents that can be used in the present disclosure include, but not limited to, natural polymers, derivatives of natural polymers, synthetic polymers and derivatives of natural polymers.

The exemplary matrices that can be used in the present disclosure include, but not limited to, natural polymers, derivatives of natural polymers, synthetic polymers and derivatives of natural polymers.

The exemplary natural polymers that can be used in the present disclosure include, but not limited to, cellulose ethers, natural gums, starches and modified products of starches.

The exemplary cellulose ethers that can be used in the present disclosure include, but not limited to, methyl cellulose, carboxymethyl cellulose, sodium carboxymethylcellulose, ethyl cellulose, hydroxyethyl cellulose, methyl 2-hydroxyethyl cellulose, hydroxypropyl cellulose and hypromellose.

The exemplary natural gums that can be used in the present disclosure include, but not limited to, acacia senegal, acacia gum, guar gum, locust bean gum, tamarind polysaccharide gum, sesbania gum, linseed gum, gleditsia sinensis lam gum, pectin, ablmoschus manihot gums, carrageenan, agar, sodium alginate, potassium alginate, gelatin, chitin, xanthan gum, β-cyclodextrin, polydextrose, gellan gum and sodium hyaluronate.

The exemplary starches and modified products of starches that can be used in the present disclosure include, but not limited to, starch, carboxymethyl starch sodium, sodium starch phosphate, hydroxypropyl starch ether, acetylated distarch phosphate, hydroxypropyl distarch phosphate, phosphated distarch phosphate, sodium starch octenyl succinate, oxystarch, acetylated distarch adipate, acid modified starch, aluminum starch octenylbutanedioate and starch acetate.

The exemplary synthetic polymers that can be used in the present disclosure include, but not limited to, acrylic resin, polyurethane and propylene glycol alginate.

The exemplary acrylic resins that can be used in the present disclosure include, but not limited to, methacrylic acid copolymer, ethyl acrylic acid copolymer, propyl acrylic copolymer and butyl acrylic acid copolymer.

The exemplary methacrylic acid copolymers that can be used in the present disclosure include, but not limited to, polymers copolymerized with methyl methacrylate or methacrylic acid and one or more of the following monomers in any ratio, wherein the exemplary monomers include, but not limited to, methyl methacrylate, methacrylic acid, and butyl methacrylate. 2-(dimethylamino) ethyl methacrylate, ethyl acrylate, 2-(trimethylammonio)ethyl 2-methylpropenoate, methyl acrylate and 2-(dimethylamino)ethyl methacrylate.

The exemplary methacrylic acid copolymers that can be used in the present disclosure include, but not limited to, butyl methacrylate/2-(dimethylamino)ethyl methacrylate/methyl methacrylate (1:2:1) copolymer, methacrylic acid/ethyl acrylate (1:1) copolymer, methacrylic acid/methyl methacrylate (1:1) copolymer, methacrylic acid/methyl methacrylate (1:2) copolymer, ethyl propenoate groups/methyl 2-methylpropenoate groups/2-(trimethylammonio)ethyl 2-methylpropenoate groups copolymer (1:2:0.2), ethyl propenoate groups/methyl 2-methylpropenoate groups/2-(trimethylammonio)ethyl 2-methylpropenoate groups copolymer (1:2:0.1), ethyl acrylate/methyl methacrylate (2:1) copolymer, and methacrylic acid/methyl acrylate/methyl methacrylate (1:1:1) copolymer.

The exemplary surfactants that can be used in the present disclosure include, but not limited to, saponin, acacia, tragacanth, gelatin, propylene monostearate, glyceryl monostearate, ethylene glycol distearate, diglyceryl monooleate, sodium lauryl sulfate, Span 20, Span 40, Span 60, Span 65, Span 80, Span 83, Span 85, potassium oleate, sodium oleate, triethanolamine oleate, lecithin, sucrose ester, poloxamer 188, Atlas G-263, Tween 20, Tween 21, Tween 40, Tween 60, Tween 61, Tween 65, Tween 80, Tween 81, Tween 85, Myrj 45, Myrj 49, Myrj 51, Myrj 52, polyethylene glycol monolaurate, polyoxyethylene monostearate, polyethylene glycol monooleate, Brij 35, Brij 30, cetomacrogol, alcohol ethoxylate, emulphor, ethoxylated hydrogenated castor oil, polyoxyethylene alkyl phenol and nonylphenoxypoly(ethyleneoxy)ethanol.

The exemplary stabilizers that can be used in the present disclosure include, but not limited to, buffer salt and amino acid.

The exemplary buffer salts that can be used in the present disclosure include, but not limited to, ammonia water, ammonium salt, sylvine, sodium salt, phthalic acid, phthalate, sulphurous acid, sulphite, citric acid, citrate, succinic acid, succinate, acetic acid, hydrochloric acid, hydrochloride, acetate, carbonic acid, carbonate, phosphoric acid, phosphate, sodium hydroxide and potassium hydroxide.

The exemplary amino acids that can be used in the present disclosure include, but not limited to, glycine, alanine, valine, leucine, isoleucine, phenylalanine, tryptophan, tyrosine, aspartic acid, asparaginate, glutamic acid, lysine, glutamine, methionine, serine, threonine, cysteine, proline, histidine and arginine.

The exemplary isosmotic adjusting agents that can be used in the present disclosure include, but not limited to, mannitol, sodium chloride, glucose, sorbitol, glycerol, polyethylene glycol and propylene glycol.

In some embodiments of the present disclosure, the microspheres comprise an active pharmaceutical ingredient, a biodegradable material and a pharmaceutically acceptable excipient.

In some embodiments of the present disclosure, the liposomes comprise an active pharmaceutical ingredient, a biodegradable material, a phosphatide, a fatty acid ester and a pharmaceutically acceptable excipient.

In some embodiments of the present disclosure, the multivesicular liposomes comprise an active pharmaceutical ingredient, a biodegradable material, a phosphatide, a fatty acid ester and a pharmaceutically acceptable excipient.

The exemplary biodegradable materials that can be used in the present disclosure include, but not limited to, PLGA (poly(lactic-co-glycolic acid)), PLA (polylactic acid), PLC (polylactide-caprolactone copolymer), PGA (polyglycolic acid), hyaluronic acid, collagen, SAIB (sucrose acetate isobutyrate), poly(orthoesters), PEG (polyethylene glycol), alginate, PCL (polycaprolactone), PCE (polycaprolactone-polyethylene glycol), PCEL (polycaprolactone-polyethylene glycol-polylactide) and PHB (poly-β-hydroxybutyrate).

The exemplary phosphatides that can be used in the present disclosure include, but not limited to, lecithin, lecithin hydrogenated, MPPC (1-myristoyl-2-palmitoyl-sn-glycero-3-phosphatidylcholine), DDPC (didecanoyl phosphatidylcholine), DLPC (dilauroyl phosphatidylcholine), DAPC (dieicosanoyl phosphatidylcholine), DMPC (dimyristoyl phosphatidylcholine), DOPC (dioleoyl phosphatidylcholine), DPPC (dipalmitoyl phosphatidylcholine), DSPC (distearoyl phosphatidylcholine), POPC (1-palmitoyl-2-oleoyl-sn-glycero-3-phosphocholine), SMPC (1-stearoyl-2-myristoyl-sn-glycero-3-phosphatidylcholine), HSPC (hydrogenated soybean phospholipids), DEPC (dierucoyl phosphatidylcholine), MPPC (1-myristoyl-2-palmitoyl-sn-glycero-3-phosphatidylcholine), MSPC (1-myristoyl-2-stearoyl-sn-glycero-3-phosphatidylcholine), PMPC (1-palmitoyl-2-myristoyl-sn-glycero-3-phosphatidylcholine), PSPC (1-palmitoyl-2-stearoyl-sn-glycero-3-phosphatidylcholine), SPPC (1-stearoyl-2-palmitoyl-sn-glycero-3-phosphatidylcholine), MOPC (1-myristoyl-2-oleoyl-sn-glycero-3-phosphatidylcholine), SOPC (1-stearoyl-2-oleoyl-sn-glycero-3-phosphocholine), PLPC (1-palmitoyl-2-lauroyl-phosphatidylcholine), DPePC chloride phosphatidylcholine (dipalmitoyl ethylphosphatidylcholine chloride salt), DPePC triflate Phosphatidylcholine (dipalmitoyl ethylphosphatidylcholine Tf salt) and mPEG-DSPE (PEGylated phospholipid).

The exemplary mPEG-DSPE (PEGylated phospholipid) that can be used in the present disclosure includes, but not limited to, mPEG-DSPE (PEGylated phospholipid), mPEG-DMPE (PEG phospholipid conjugate), DSPE-PEG-MAL (maleimide functionalized PEG lipid), DSPE-PEG-COOH (carboxylic acid functionalized PEG lipid), DSPE-PEG-NH2 (amine functionalized PEG lipid), DSPE-PEG-Biotin (biotin functionalized PEG lipid) and mPEG-Cholesterol (PEG reagents for liposome drug delivery system).

The exemplary fatty acid esters that can be used in the present disclosure include, but not limited to, triolein, glycerol trioleate, tricaprylin, oleic acid and glyceryl tri(2-ethylhexanoate).

In some embodiments of the present disclosure, the nanocrystals comprise nanoparticles of an active pharmaceutical ingredient and a pharmaceutically acceptable excipient.

The exemplary active pharmaceutical ingredients that can be used in the present disclosure include, but not limited to, neostigmine, pyridostigmine, edrophonium chloride, ambenonium chloride, physostigmine, demacarium bromide and galanthamine.

In some embodiments of the present disclosure, the ophthalmic injectable sustained-release formulation can be sustained-release in vivo in one week.

In some embodiments of the present disclosure, the ophthalmic injectable sustained-release formulation can be sustained-release in vivo in two weeks.

In some embodiments of the present disclosure, the ophthalmic injectable sustained-release formulation can be sustained-release in vivo in one to three months.

In some embodiments of the present disclosure, the ophthalmic injectable sustained-release formulation can be sustained-release in vivo in six months.

In some embodiments of the present disclosure, the ophthalmic injectable sustained-release formulation can be sustained-release in vivo in more than six months.

In another aspect, the disclosure relates to a process for preparing an ophthalmic injectable sustained-release formulation comprising a delivery system and a pharmaceutically acceptable excipient,

wherein the delivery system is selected from the group consisting of microspheres, microcapsules, microparticles, liposomes, multivesicular liposomes, nanocrystals and nanoparticles; and

wherein the process comprises

preparing the microspheres via emulsion solvent evaporation, double emulsion solvent evaporation, emulsification-chemical cross-linking method, emulsion-direct heat cross-linking method, spray drying, phase separation, SCF (supercritical fluid), ultrasonic atomization, electro spraying, hot melt extrusion, or polymer-alloys method.

In yet another aspect, the disclosure relates to a process for preparing an ophthalmic injectable sustained-release formulation comprising a delivery system and a pharmaceutically acceptable excipient,

wherein the delivery system is selected from the group consisting of microspheres, microcapsules, microparticles, liposomes, multivesicular liposomes, nanocrystals and nanoparticles; and

wherein the process comprises

preparing the liposomes via thin-film hydration, sonication, membrane extrusion, REV (reverse phase evaporation), ether injection, ethanol injection, freezing thawing, French press method, multiple emulsion, SRPE (supercritical fluid reverse phase evaporation), freeze drying of double emulsions, or ion gradient.

In still another aspect, the disclosure relates to a process for preparing an ophthalmic injectable sustained-release formulation comprising a delivery system and a pharmaceutically acceptable excipient,

wherein the delivery system is selected from the group consisting of microspheres, microcapsules, microparticles, liposomes, multivesicular liposomes, nanocrystals and nanoparticles; and

wherein the process comprises

preparing the multivesicular liposomes via double emulsion solvent evaporation.

In yet another aspect, the disclosure relates to a method for treating and preventing oculopathy, comprising administering an ophthalmic injectable sustained-release formulation to a subpalpebral conjunctiva plane just superior to a superior tarsal border across a horizontal width of an upper eyelid of an affected eye of a subject in need thereof,

wherein ophthalmic injectable sustained-release formulation comprises a delivery system and a pharmaceutically acceptable excipient, and

wherein the delivery system is selected from the group consisting of microspheres, microcapsules, microparticles, liposomes, multivesicular liposomes, nanocrystals and nanoparticles.

The exemplary oculopathy that can be treated or prevented by the method of the present disclosure includes, but not limited to, ocular myasthenia gravis (OMG), blepharospasm, dermatolysis palpebrarum, involutional, myogenic, neurogenic, and congenital ptosis, trichiasis and eyelid tumors.

In some embodiments of the present disclosure, the method comprises everting an upper eyelid to expose a palpebral conjunctiva.

In some embodiments of the present disclosure, the method comprises applying a drop of ophthalmic topical anesthetic to the affected eye.

In some embodiments of the present disclosure, the method comprises applying topical anesthetic to the palpebral conjunctiva.

In some embodiments of the present disclosure, the method comprises prior to everting an upper eyelid to expose a palpebral conjunctiva cleaning the surgical area in the standard, sterile, oculoplastic and ophthalmic fashion with betadine® swabs.

In some embodiments of the present disclosure, the method comprises after applying the ophthalmic injectable sustained-release formulation cleaning the surgical area with sterile saline.

In some embodiments of the present disclosure, the method for treating and preventing oculopathy, comprising

applying a drop of ophthalmic topical anesthetic to an affected eye;

cleaning the surgical area in a standard, sterile, oculoplastic and ophthalmic manner with betadine® swabs;

everting an upper eyelid to expose a palpebral conjunctiva;

applying topical anesthetic to the palpebral conjunctiva;

applying an ophthalmic injectable sustained-release formulation in a subpalpebral conjunctiva plane just superior to a superior tarsal border to variable extents across a horizontal width of the upper eyelid;

checking the surgical area to ensure appropriate hemostasis;

returning the eyelid to its normal anatomic position; and

cleaning the surgical area with sterile saline;

wherein the ophthalmic injectable sustained-release formulation comprises a delivery system and a pharmaceutically acceptable excipient, and

wherein the delivery system is selected from the group consisting of microspheres, microcapsules, microparticles, liposomes, multivesicular liposomes, nanocrystals and nanoparticles.

In some embodiments of the present disclosure, the method for treating and preventing oculopathy further comprises injecting a subconjunctival anesthetic in the plane just under the palpebral conjunctiva.

In some embodiments of the present disclosure, the method for treating and preventing oculopathy, comprising:

applying a drop of ophthalmic topical anesthetic to an affected eye;

cleaning the surgical area in a standard, sterile, oculoplastic and ophthalmic manner with betadine® swabs;

everting an upper eyelid to expose a palpebral conjunctiva;

applying topical anesthetic to the palpebral conjunctiva;

injecting a subconjunctival anesthetic in the plane just under the palpebral conjunctiva;

applying an ophthalmic injectable sustained-release formulation in a subpalpebral conjunctiva plane just superior to a superior tarsal border to variable extents across a horizontal width of the upper eyelid;

checking the surgical area to ensure appropriate hemostasis;

returning the eyelid to its normal anatomic position; and

cleaning the surgical area with sterile saline;

wherein the ophthalmic injectable sustained-release formulation comprises a delivery system and a pharmaceutically acceptable excipient, and

wherein the delivery system is selected from the group consisting of microspheres, microcapsules, microparticles, liposomes, multivesicular liposomes, nanocrystals and nanoparticles.

### EXAMPLES

Although anyone skilled in the art is capable of preparing the formulations of the present disclosure according to the general techniques disclosed above, more specific details on synthetic techniques for formulations of the present disclosure are provided elsewhere in this specification for convenience. Again, all reagents and reaction conditions employed in synthesis are known to those skilled in the art and are available from ordinary commercial sources.

### Example 1

Preparation of Neostigmine MVL (Multivesicular Liposomes)

| Formula | Ingredient | g/Batch | |
|---|---|---|---|
| Part I | Neostigmine | 0.53 | |
| | potassium hydrogen phosphate | 0.005 | |
| | monopotassium phosphate | 0.11 | |
| | Purified Water* | 10 | mL |
| Part II | Glucose | 16 | |
| | L-Lysine | 3.28 | |
| | Purified Water* | 500 | mL |
| Part III | DPPC (Dipalmitoyl Phosphatidylcholine) | 0.33 | |
| | DSPC (Distearoyl Phosphatidylcholine) | 0.04 | |
| | Tricaprylin | 0.08 | |
| | Cholesterol | 0.19 | |
| | Dichloromethane* | 14 | mL |
| Part IV | Sodium Chloride | 9 | |
| | Purified Water* | 1,000 | mL |
| Part V | Glucose | 16 | |
| | Purified Water* | 500 | mL |

Note:
*means does not exist in the final product

Solution A: Required amount of Neostigmine, potassium hydrogen phosphate, and monopotassium phosphate were weighed and dissolved into 10 mL purified water. (Labeled as Part I)

Solution B: Required amount of Glucose and L-Lysine were weighed and dissolved into 500 mL purified water. (Labeled as Part II)

Solution C: Required amount of excipients (DPPC, DSPC, Tricaprylin and cholesterol) were weighed and added into 14 mL dichloromethane, then vortexed for about 5 min to dissolve. (Labeled as Part III)

Solution D: Required amount of Sodium Chloride was weighed and into 1,000 mL purified water. (Labeled as Part IV)

W/O (water-in-oil) emulsion: The solution A was added into Solution C, and subjected to mixing at 2,500 rpm in an omni-mixer homogenizer for 10 minutes.

W/O/W (water-in-oil-in-water) double emulsion: 2 mL solution B was added into the W/O emulsion and the mixture was sheared for 1 minute, then the mixture was transferred into 10.5 mL of solution B, and subjected to mixing for 60 seconds at 6,000 rpm.

MVL particles: The resulting water-in-oil-in-water double emulsion was transferred immediately to 135 mL Solution B. The dichloromethane was evaporated under a constant flow (3 L/min) of nitrogen gas for 5 minutes with 2,000 rpm magnetic stirring under purified water bath at −4° C. to form MVL particles in suspension.

The MVL particles were isolated by centrifugation at 8,000 rpm for 20 minutes, then washed twice with Solution D, and freeze-dried to obtain powdery MVL.

### Example 2

Preparation of Pyridostigmine Microspheres

| Ingredient | g/Batch |
|---|---|
| Pyridostigmine | 0.811 |
| PLGA | 10 |
| PVA | 5.03 |
| methyl alcohol* | 15 |
| Dichloromethane* | 100 |
| purified water* | 1000 |

Note:
*means does not exist in the final product

Solution A: Required amount of Pyridostigmine was weighed and dissolved in methyl alcohol.

Solution B: Required amount of PLGA was weighed and dissolved in dichloromethane.

Solution A and solution B were mixed in a ratio of 10:1, and subjected to emulsification in a high shear emulsifier (6,500 rpm, 3 min) so as to obtain a W/O primary emulsion.

The primary emulsion was added into 1,000 mL of a 0.5% PVA solution at 6° C. under homogenization at 1,800 rpm, and then it was homogeneously emulsified for 2 min to obtain a W/O/W double emulsion.

The double emulsion was stirred to volatilize and remove the organic solvent. The residue was washed and freeze-dried to obtain powdery microspheres.

### Example 3

Preparation of Neostigmine Microspheres

| | Ingredient | g/Batch | |
|---|---|---|---|
| Part I | Neostigmine | 0.3 | |
| | PLGA (50:50, Mw = 20000) | 5 | |
| | glacial acetic acid* | 50 | ml |
| Part II | normal heptane* | 200 | ml |
| | silicone oil* | 200 | ml |
| Part III | normal heptane* | 1,800 | ml |
| | ethyl alcohol* | 1,800 | ml |

| | | |
|---|---|---|
| Part IV | normal heptane:ethyl alcohol 1:1* | q.s. |

Note:
*means does not exist in the final product

Required amount of Neostigmine and PLGA (part I) were weighed and dispersed in glacial acetic acid to form polymer solution.

Flocculant: Required amount of normal heptane and silicone oil (part II) were weighed and mixed.

The polymer solution was homogenized by homogenizer, and the flocculant was added into polymer solution under homogenizing. Primary microspheres were obtained.

The normal heptane and ethyl alcohol labeled as part III were mixed, and the primary microspheres were transferred in it to stir for 1-2 hours under 20° C.

The residue was washed by solution labeled as part IV and freeze-dried for 24 hours under 4° C. to obtain powdery microspheres.

### Example 4

Preparation of Galanthamine Microspheres

| Ingredient | g/Batch |
|---|---|
| Galanthamine | 0.3 |
| PLGA (75:25, Mw = 20,000) | 3 |
| glacial acetic acid* | 15 |

Note:
*means does not exist in the final product

Required amount of Galanthamine and PLGA were weighed in glacial acetic acid, and dissolved completely under ultrasonic oscillation.

The mixed solution was spray dried under following parameters: inlet temperature 50° C., outlet temperature 35° C., flow rate 30%, spray pressure 50%. The microspheres were obtained.

### Example 5

Preparation of Galanthamine Microspheres

| Ingredient | g/Batch |
|---|---|
| Galanthamine | 0.3 |
| PLGA (65:35, Mw = 20,000) | 3 |

Required amount of Galanthamine and PLGA were weighed and mixed by blender for 10 minutes.

The mixture was added into twin-screw extruder and extruded under following parameters: extrusion temperature 160° C., speed 160 rpm. The extrudate was cooled and obtained.

The extrudate was cut into 3 mm short strips by granulator, and smashed.

The microspheres sieved through 120 mesh sieve were collected.

### Example 6

Preparation of Neostigmine Injectable Gel

| Ingredient | g/Batch |
|---|---|
| Neostigmine powdery Multivesicular | 1 |
| Liposome prepared in Example 1 | |
| sodium hyaluronate | 10 |
| hypromellose | 2 |

The Neostigmine powdery Multivesicular Liposome prepared in Example 1, sodium hyaluronate and hypromellose were weighed and stirred by stirrer for 5 minutes to form Neostigmine injectable gel.

### Example 7

Preparation of Pyridostigmine Suspension for Injectable

| Ingredient | g/Batch |
|---|---|
| Pyridostigmine powdery microspheres | 1.5 |
| prepared in Example 2 | |
| hydroxypropyl cellulose | 2 |
| carboxymethylcellulose sodium | 8 |

The Pyridostigmine powdery microspheres prepared in Example 2, hydroxypropyl cellulose and carboxymethylcellulose sodium were weighed and stirred by stirrer for 5 minutes to form Pyridostigmine Suspension for injectable.

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

What is claimed is:

1. A method for treating and preventing oculopathy, comprising administering an ophthalmic injectable sustained-release formulation to a subpalpebral conjunctiva plane just superior to a superior tarsal border across a horizontal width of an upper eyelid of an affected eye of a subject in need thereof, wherein the ophthalmic injectable sustained-release formulation comprises a delivery system and a pharmaceutically acceptable excipient, wherein the delivery system is selected from the group consisting of microspheres, microcapsules, microparticles, liposomes, multivesicular liposomes, nanocrystals and nanoparticles.

2. The method of claim 1, wherein the oculopathy is selected from the group consisting of ocular myasthenia gravis (OMG), blepharospasm, dermatolysis palpebrarum, involutional, myogenic, neurogenic, and congenital ptosis, trichiasis and eyelid tumors.

3. The method of claim 1, wherein the method comprises everting an upper eyelid to expose a palpebral conjunctiva.

4. The method of claim 1, wherein the method comprises applying a drop of ophthalmic topical anesthetic to the affected eye.

5. The method of claim 1, wherein the method comprises applying topical anesthetic to the palpebral conjunctiva.

6. The method of claim 1, wherein the method comprises injecting a subconjunctival anesthetic in the plane just under palpebral conjunctiva.

7. The method of claim 1, wherein the method comprises
applying a drop of ophthalmic topical anesthetic to an affected eye;
cleaning the surgical area in a standard, sterile, oculoplastic and ophthalmic manner with betadine® swabs;
everting an upper eyelid to expose a palpebral conjunctiva;
applying topical anesthetic to the palpebral conjunctiva;
injecting a subconjunctival anesthetic in the plane just under the palpebral conjunctiva;
applying the ophthalmic injectable sustained-release formulation in a subpalpebral conjunctiva plane just superior to a superior tarsal border to variable extents across a horizontal width of the upper eyelid;
checking the surgical area to ensure appropriate hemostasis;
returning the eyelid to its normal anatomic position; and
cleaning the surgical area with sterile saline.

8. The method of claim 1, wherein the ophthalmic injectable sustained-release formulation is in the form of solution, suspension, or gel.

9. The method of claim 1, wherein the microspheres comprise an active pharmaceutical ingredient, a biodegradable material and a pharmaceutically acceptable excipient.

10. The method of claim 1, wherein the liposomes comprise an active pharmaceutical ingredient, a biodegradable material, a phosphatide, a fatty acid ester and a pharmaceutically acceptable excipient.

11. The method of claim 1, wherein the multivesicular liposomes comprise an active pharmaceutical ingredient, a biodegradable material, a phosphatide, a fatty acid ester and a pharmaceutically acceptable excipient.

12. The method of claim 1, wherein the nanocrystals comprise nanoparticles of active pharmaceutical ingredient and a pharmaceutically acceptable excipient.

13. The method of claim 9, wherein the active pharmaceutical ingredient is selected from the group consisting of neostigmine, pyridostigmine, edrophonium chloride, ambenonium chloride, physostigmine, demacarium bromide and galanthamine.

14. The method of claim 10, wherein the active pharmaceutical ingredient is selected from the group consisting of neostigmine, pyridostigmine, edrophonium chloride, ambenonium chloride, physostigmine, demacarium bromide and galanthamine.

15. The method of claim 11, wherein the active pharmaceutical ingredient is selected from the group consisting of neostigmine, pyridostigmine, edrophonium chloride, ambenonium chloride, physostigmine, demacarium bromide and galanthamine.

16. The method of claim 11, wherein the active pharmaceutical ingredient is selected from the group consisting of neostigmine, pyridostigmine, edrophonium chloride, ambenonium chloride, physostigmine, demacarium bromide and galanthamine.

17. The method of claim 12, wherein the active pharmaceutical ingredient is selected from the group consisting of neostigmine, pyridostigmine, edrophonium chloride, ambenonium chloride, physostigmine, demacarium bromide and galanthamine.

18. The method of claim 1, wherein the ophthalmic injectable sustained-release formulation is sustained-release in one week, two weeks, one to three months, six months, or longer.

19. An ophthalmic injectable sustained-release formulation, comprising a delivery system and a pharmaceutically acceptable excipient, wherein the delivery system is selected from the group consisting of microspheres, microcapsules, microparticles, liposomes, multivesicular liposomes, nanocrystals and nanoparticles.

20. The ophthalmic injectable sustained-release formulation of claim 19, wherein the ophthalmic injectable sustained-release formulation is in the form of solution, suspension, or gel.
